(19) United States
(12) Patent Application Publication
(10) Pub. No.: US 2006/0217437 A1
(43) Pub. Date: Sep. 28, 2006

(54) TGF-beta modulators and methods for using the same

(76) Inventor: James K. Burmester, Marshfield, WI (US)

Correspondence Address: FAEGRE & BENSON LLP;PATENT DOCKETING, 2200 WELLS FARGO CENTER, 90 SOUTH 7TH STREET, MINNEAPOLIS, MN, 55402-3901, US

(21) Appl. No.: 11/384,113

(22) Filed: Mar. 17, 2006

Related U.S. Application Data
(60) Provisional application No. 60/663,312, filed on Mar. 17, 2005.

(51) Int. Cl. A61K 31/353; A61K 31/473; A61K 31/382

(52) U.S. Cl. 514/454; 514/297

(57) ABSTRACT

The present invention provides a method of modulating TGF-β activity in a subject. Methods of the present invention comprise administering to the subject an effective amount of a compound of the formula: 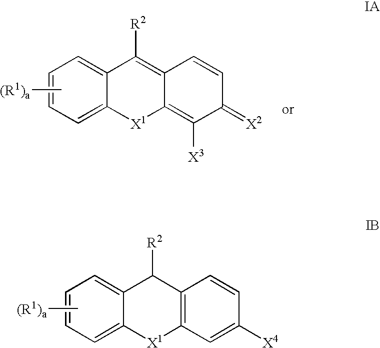 or a salt, prodrug, tautomer, hydrate, solvate, or stereoisomer thereof, wherein a is an integer from 0 to 4;
each R¹ is independently hydroxy, alkoxy, halide, alkyl, cyano, nitro, amino, monoalkylamino, dialkylamino, or carboxy;
R² is aryl, cycloalkyl, alkenyl, or alkyl, each of which is optionally substituted;
each of X¹ and X² is independently O, S or NR, wherein
   R³is hydrogen or alkyl;
X³ is hydrogen, alkoxy, alkyl, hydroxy, halide, amino, monoalkylamino, or dialkylamino; and
   X⁴ is alkoxy, hydroxy, halide, amino, monoalkylamino, or dialkylamino.

### CROSS-REFERENCE TO RELATED APPLICATIONS

This application claims the priority benefit of U.S. Provisional Application No. 60/663,312, filed Mar. 17, 2005, which is incorporated herein by reference in its entirety.

### FIELD OF THE INVENTION

The present invention relates to TGF-β modulators and methods for using the same.

### BACKGROUND OF THE INVENTION

Transforming growth factor β (i.e., TGF-β) activates both antiproliferative and tumor-promoting signaling cascades. Three mammalian TGF-β isoforms have been identified (TGF-βI, -βII, and -βIII). TGF-β production promotes tumor progression while its blockade enhances antitumor activity. Blockade of TGF-β enhances antitumor immune responses and inhibits metastasis. In addition, excessive TGF-β production results in other various ailments, including fibrotic diseases such as glomerulonephritis, diabetic nephropathy, hepatic fibrosis, pulmonary fibrosis, arterial scleroderma, excessive scarring, hyperplasia, and restenosis.

Conventional approaches for modulating TGF-β signaling include use of antibodies to block binding of TGF-β ligand to the heteromeric receptor complex, and intracellular inhibition of phosphorylating enzyme, e.g., type I TGF-β receptor kinase, with small-molecules. Yingling et al., Nature Reviews, 2004, 3, 1011-1022. Unfortunately, there are a variety of undesirable side effects associated with current methods for modulating TGF-β.

For example, antibodies are made of proteins. Proteins are generally not suitable for oral administration. In addition, proteins often elicit an immune response, and therefore are not well tolerated by patients. While small molecules generally do not have the same immune response eliciting problems as antibodies, conventional small molecule treatment of TGF-β disorder targets kinase enzymes. Since the kinases are involved in many different intracellular activities, inhibition of kinases often results in a cascade of other undesirable side effects.

Therefore, there is a need for modulating TGF-β activities with potentially reduced side effects.

### SUMMARY OF THE INVENTION

One aspect of the present invention is directed to a method of modulating TGF-β activity in a subject. In this particular aspect, methods of the present invention comprise administering to the subject an effective amount of a compound of the formula: 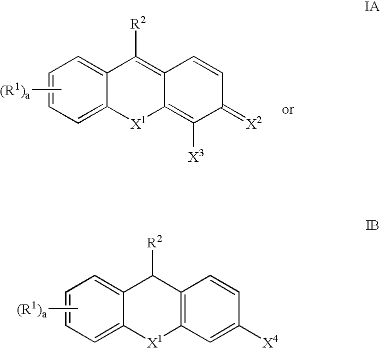
or a salt, prodrug, tautomer, hydrate, solvate, or stereoisomer thereof, wherein
a is an integer from 0 to 4;
each R¹ is independently hydroxy, alkoxy, halide, alkyl, cyano, nitro, amino, monoalkylamino, dialkylamino, or carboxy;
R² is aryl, cycloalkyl, alkenyl, or alkyl, each of which is optionally substituted;
each of X¹ and X² is independently O, S or NR³,
wherein
   R³is hydrogen or alkyl;
X³ is hydrogen, alkoxy, alkyl, hydroxy, halide, amino, monoalkylamino, or dialkylamino; and
X⁴ is alkoxy, hydroxy, halide, amino, monoalkylamino, or dialkylamino.

Another aspect of the present invention is directed to a method of treating a TGF-mediated disease. Such methods comprise administering to a patient suffering from a TGF-mediated disease a therapeutically effective amount of a TGF-β antagonist. Exemplary TGF-mediated diseases that can be treated by methods of the present invention include cancer, glomerulonephritis, diabetic nephropathy, hepatic fibrosis, pulmonary fibrosis, arterial scleroderma, excessive scarring, hyperplasia, restenosis, scleroderma, dermal scarring, as well as other TGF-mediated diseases known to one skilled in the art.

Yet another aspect of the present invention provides a method for stimulating a patient's immune system. In this particular aspect of the present invention, the method comprises administering a therapeutically effective amount of a TGF-β antagonist to the patient.

Still another aspect of the present invention provides a method for reducing the risk of cancer in a subject. This method comprises administering a therapeutically effective amount of a TGF-β modulator to the subject to significantly reduce the TGF-β activity.

### BRIEF DESCRIPTION OF THE DRAWINGS

FIGS. 1-1C shows some of the compounds that were assayed according to the procedure of Example 1;

FIG. 2A is a graph showing luciferase activity of some of the compounds assayed in the absence of TGF-β1;

FIG. 2B is a graph showing luciferase activity of some of the compounds assayed in the presence of TGF-β1;

FIG. 3 is a graph showing the effect of some of the compounds on the growth of DLD-1 cells;

FIG. 4A is a graph showing the ability of compound NSC 119889 to block cell growth inhibition of TGF-β1;

FIG. 4B is a graph showing the ability of compound NSC 119915 to block cell growth inhibition of TGF-β1;

FIG. 4C is a graph showing the ability of compound NSC 119911 to block cell growth inhibition of TGF-β1;

FIG. 4D is a graph showing the effect of control compound NSC 306960 on the cell growth inhibition of TGF-β1;

FIG. 5A is a graph showing CHO cell growth stimulation by 1 μL of compound NSC 119889;

FIG. 5B is a graph showing CHO cell growth stimulation by 2 μL of compound NSC 119889;

FIG. 6 is a graph showing a binding competition curve for various compounds vs. TGF-β1 for soluble TβRII;

FIG. 7 is a graph showing T47D breast cancer cell growth in the presence of activin under various conditions; and

FIG. 8 is a Northern blot from incubation of mink lung epithelial cells under various conditions.

### DETAILED DESCRIPTION OF THE INVENTION

### DEFINITIONS

The term “alkenyl” means a linear monovalent hydrocarbon moiety of two to six carbon atoms or a branched monovalent hydrocarbon moiety of three to six carbon atoms, containing at least one double bond, e.g., ethenyl, propenyl, and the like. Alkenyl groups may optionally be substituted with one or more of the substituents, each of which is independently selected from the group: halide, cyano, —C(═O)R (where R is hydrogen, alkyl, haloalkyl, amino, monoalkylamino, dialkylamino, hydroxy, or alkoxy), amino (—NH₂), monoalkylamino (—NHR^{a}, where R^{a} is unsubstituted alkyl), and dialkylamino (—NR^{b}, where each R^{b} is independently unsubstituted alkyl).

“Alkyl” refers to a saturated linear monovalent hydrocarbon moiety of one to twelve, preferably one to six, carbon atoms or a saturated branched monovalent hydrocarbon moiety of three to twelve, preferably three to six, carbon atoms. Exemplary alkyl group include, but are not limited to, methyl, ethyl, n-propyl, 2 propyl, tert-butyl, pentyl, and the like. Alkyl groups may optionally be substituted with one or more of the substituents, each of which is independently selected from the group: halide (in which case it may be referred to as “haloalkyl”), cyano, —C(═O)R (where R is hydrogen, alkyl, haloalkyl, amino, monoalkylamino, dialkylamino, hydroxy, or alkoxy), amino (—NH₂), monoalkylamino (—NHR^{a}, where R^{a} is unsubstituted alkyl), and dialkylamino (—NR^{b}, where each R^{b} is independently unsubstituted alkyl).

“Alkylene” refers to a saturated linear saturated divalent hydrocarbon moiety of one to twelve, preferably one to six, carbon atoms or a branched saturated divalent hydrocarbon moiety of three to twelve, preferably three to six, carbon atoms. Exemplary alkylene groups include, but are not limited to, methylene, ethylene, propylene, butylene, pentylene, and the like. Alkylene groups may optionally be substituted with one or more substituents that are disclosed herein in reference to the alkyl group.

“Aryl” refers to a monovalent mono-, bi- or tricyclic aromatic hydrocarbon moiety of 6 to 15 ring atoms which is optionally substituted with one or more substituents within the ring structure. Preferred substituents for the aryl group include alkyl, haloalkyl, halo, nitro, cyano, cycloalkyl, heterocyclyl, —OR (where R is hydrogen, alkyl or haloalkyl, -(alkylene)ₙ-COOR (where n is 0 or 1 and R is hydrogen, alkyl, or haloalkyl), or -(alkylene)ₙ-CONR^{a}R^{b} (where n is 0 or 1, and each of R^{a} and R^{b} is independently hydrogen, alkyl, cycloalkyl, cycloalkylalkyl, aryl, aralkyl, or R^{a} and R^{b} together with the nitrogen atom to which they are attached form a heterocyclyl ring). More specifically the term aryl includes, but is not limited to, phenyl, 1-naphthyl, and 2-naphthyl, each of which may be optionally substituted.

“Chiral center” (i.e., stereochemical center, stereocenter, or stereogenic center) refers to an asymmetrically substituted atom, e.g., a carbon atom to which four different groups are attached. The ultimate criterion of a chiral center, however, is nonsuperimposability of its mirror image.

“Cycloalkyl” refers to a non-aromatic, saturated or unsaturated, monovalent mono-, bi-, or tricyclic hydrocarbon moiety of three to twelve ring carbons including bridged ring moieties. The cycloalkyl can be optionally substituted with one or more, preferably one, two, or three, substituents, where each substituent is independently selected from the group consisting of alkyl, haloalkyl, halide, cyano, or —C(═O)R (where R is hydrogen, alkyl, haloalkyl, amino, monoalkylamino, dialkylamino, hydroxy, or alkoxy). Exemplary cycloalkyl groups include, but are not limited to, cyclopropyl, cyclohexyl, cyclohexenyl, bicyclo[2.2.1]hept-2-ene, and the like, each of which may be optionally substituted.

“Cycloalkylalkyl” refers to a moiety of the formula —R^{a}R^{b} where R^{a} is an alkylene group and R^{b} is a cycloalkyl group as defined herein.

The terms “halo,” “halogen” and “halide” are used interchangeably herein and refer to fluoro, chloro, bromo, or iodo.

“Haloalkyl” refers to an alkyl group as defined herein in which one or more hydrogen atom is replaced by same or different halo atoms. The term “haloalkyl” also includes perhalogenated alkyl groups in which all alkyl hydrogen atoms are replaced by halogen atoms. Exemplary haloalkyl groups include, but are not limited to, —CH₂Cl, —CF₃, —CH₂CF₃, —CH₂CCl₃, and the like.

“Heterocyclyl” means a non-aromatic monocyclic moiety of three to eight ring atoms in which one or two ring atoms are heteroatoms selected from N, O, or S(O)ₙ (where n is an integer from 0 to 2), the remaining ring atoms being C, where one or two C atoms can optionally be a carbonyl group. The heterocyclyl ring can be optionally substituted with one or more, preferably one, two, or three, substituents. When two or more substituents are present in a heterocyclyl group, each substituent is independently selected. Preferred substituents for heterocyclyl group include, but are not limited to, alkyl, haloalkyl, heteroalkyl, halo, nitro, cyano, acyl, —C(═O)R (where R is hydrogen, alkyl, haloalkyl, amino, monoalkylamino, dialkylamino, hydroxy, or alkoxy). More specifically the term heterocyclo includes, but is not limited to, tetrahydropyranyl, piperidino, piperazino, morpholino and the like.

“Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino, and the like.

“Modulation” refers to a change in the level or magnitude of an activity or process. The change can be either an increase or a decrease. For example, modulation of TGF-β activity includes both increase and decrease in TGF-β activity. Modulation can be assayed by determining any parameter that is indirectly or directly affected by the TGF-β activity. Such parameters include, but are not limited to, cell growth, cell differentiation, and cell morphogenesis.

“Pharmaceutically acceptable excipient” refers to an excipient that is useful in preparing a pharmaceutical composition that is generally safe, non-toxic and neither biologically nor otherwise undesirable, and includes excipient that is acceptable for veterinary use as well as human pharmaceutical use.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The terms “pro-drug” and “prodrug” are used interchangeably herein and refer to a pharmacologically substantially inactive derivative of a parent drug molecule that requires biotransformation, either spontaneous or enzymatic, within the organism to release the active drug. Prodrugs are variations or derivatives of the compounds of the present invention which have groups cleavable under metabolic conditions. Prodrugs become the compounds of the present invention, which are pharmaceutically active in vivo, when they undergo solvolysis under physiological conditions or undergo enzymatic degradation. Prodrugs may require more than one biotransformation steps to release the active drug within the organism. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives that are well known to one skilled in the art, such as, but not limited to, esters prepared by reaction of the parent acids with a suitable alcohol, or amides prepared by reaction of the parent acid compound with an amine, or basic groups reacted to form an acylated base derivative. Moreover, the prodrugs of the present invention may be combined with other features herein taught to enhance bioavailability. For example, a compound of the present invention having free amino, amido, hydroxy or carboxylic groups can be converted into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of the invention. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds wherein carbonates, carbamates, amides and alkyl esters which are covalently bonded to the above substituents of a compound of the invention through the carbonyl carbon prodrug sidechain.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

“Corresponding protecting group” means an appropriate protecting group corresponding to the heteroatom (i.e., N, O, P or S) to which it is attached.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

As used herein, the term “TGF-mediated disorder” refers to any disorder mediated by the production or non-production of TGF-β.

When describing a disease (or disorder), the terms “treating” or “treatment” includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

When describing a chemical reaction, the terms “treating”, “reacting” and “contacting” are used interchangeably herein, and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

As used herein, the terms “those defined above” and “those defined herein” when referring to a variable incorporates by reference the broad definition of the variable as well as any narrow and/or preferred, more preferred and most preferred definitions, if any.

Compounds of the Present Invention

Some aspects of the present invention are directed to a method of modulating TGF-β activity in a subject. Current drug therapies utilizing small molecules are designed to modulate TGF-β activity by targeting the TGF-β signaling pathway. Typically, these methods involve intracellular inhibition of type I TGF-β receptor kinase. Thus, most known small molecules do not target the TGF-β receptors directly. Without being bound by any theory, it is believed that compounds of the present invention modulate TGF-β activity by binding to the TGF-β receptor directly or by binding to TGF-β protein(s) rather than inhibiting TGF-β receptor kinase.

Some methods of the present invention comprise a compound of the formula: 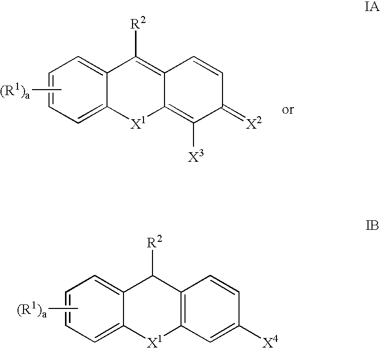
where
a is an integer from 0 to 4;
each R¹ is independently hydroxy, alkoxy, halide, alkyl, haloalkyl, cyano, nitro, amino, monoalkylamino, dialkylamino, or carboxy;
R² is aryl, cycloalkyl, alkenyl, or alkyl, each of which is optionally substituted; each of X¹ and X² is independently O, S or NR³,
wherein
   R³is hydrogen or alkyl;
X³ is hydrogen, alkoxy, alkyl, haloalkyl, hydroxy, halide, amino, monoalkylamino, or dialkylamino; and
X⁴ is alkyl, alkoxy, haloalkyl, haloalkoxy, hydroxy, halide, amino, monoalkylamino, or dialkylamino.

In one example, a is 1 or 2.

Some of the compounds of the present invention include those where each R¹ is independently hydroxy or alkoxy. Preferably, R¹ is hydroxy.

Other examples of compounds of the present invention include compounds where X¹ is O.

Still other examples of compounds of the present invention include compounds where X² is O.

Some of the compounds of the present invention include compounds where X³ is hydroxy or alkoxy. Preferably X³ is hydroxy.

Another example of compounds of the present invention include compounds where R² is substituted aryl (e.g., substituted phenyl), substituted cycloalkyl (e.g., substituted cyclohexyl and substituted bicyclo[2.2.1]hept-2-en-6-yl), substituted alkenyl (e.g., 2-carboxyethenyl), or substituted alkyl (e.g., 2-carboxyethyl). In one particular example, R² is a penta-substituted phenyl such as 2-carboxy,3,4,5,6-tetrabromophenyl. In another particular example, R² is a substituted cyclohexyl, such as 2-carboxy-cyclohexyl, or a substituted bicyclo[2.2.1]hept-2-en-6-yl, such as 5-carboxy-7-methyl bicyclo[2.2.1]hept-2-en-6-yl. Yet another particular example of compounds of the present invention is a substituted ethenyl, for example, 2-carboxyethenyl. Still another particular example of compounds of the present invention includes a substituted alkyl, e.g., 2-carboxyethyl.

Still some of the compounds of the present invention include compounds where X⁴ is hydroxy or alkoxy. Preferably X⁴ is hydroxy.

Still further, combinations of the exemplary functional groups described herein form other preferred embodiments. In this manner a variety of specific compounds are embodied within the scope of the present invention. Some of the specific compounds of the present invention include:

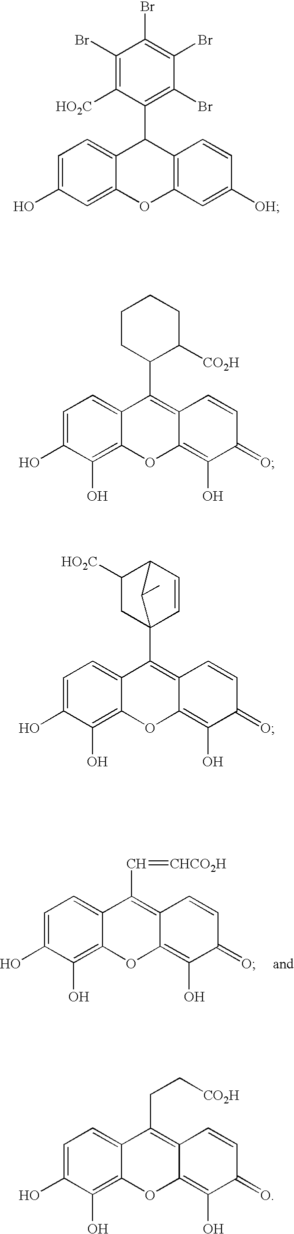

The compounds of the present invention can exist in unsolvated forms as well as solvated forms, including hydrated forms. In general, the solvated forms, including hydrated forms, are equivalent to unsolvated forms and are intended to be encompassed within the scope of the present invention. In addition to the compounds described above, the compounds of the present invention include all tautomeric forms. Furthermore, the present invention also includes all prodrug forms of the compounds and all stereoisomers whether in a pure chiral form or a racemic mixture or other form of mixture as well as geometric stereoisomers such as (E)- and (Z)-olefins.

The compounds of the present invention are capable of further forming pharmaceutically acceptable acid addition salts. All of these forms are within the scope of the present invention.

Pharmaceutically acceptable acid addition salts of the compounds of the present invention include salts derived from inorganic acids such as hydrochloric, nitric, phosphoric, sulfuric, hydrobromic, hydriodic, phosphorous, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge et al., J. of Pharmaceutical Science, 1977, 66, 1-19).

The acid addition salts of the basic compounds can be prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms somewhat in certain physical properties such as solubility in polar solvents, but otherwise the salts are equivalent to their respective free base for purposes of the present invention.

Pharmaceutically acceptable base addition salts can be formed with metal ions or amines, such as alkali and alkaline earth metal ions or organic amines. Examples of metal ions which are used as cations include sodium, potassium, magnesium, calcium, and the like. Examples of suitable amines are N,N′-dibenzylethylenediamine, chloroprocaine, choline, diethanolamine, ethylenediamine, N-methylglucamine, and procaine.

The base addition salts of acidic compounds can be prepared by contacting the free acid form with a sufficient amount of the desired base to produce the salt in the conventional manner. The free acid form can be regenerated by contacting the salt form with an acid and isolating the free acid in the conventional manner. The free acid forms may differ from their respective salt forms somewhat in certain physical properties such as solubility in polar solvents, but otherwise the salts are equivalent to their respective free acid for purposes of the present invention.

The compounds of the present invention can be prepared by a variety of methods which will become apparent to those skilled in the art of organic chemistry. Suitable synthetic methods can readily be formulated given appropriate consideration to protection and deprotection of reactive functional groups by methods standard to the art of organic chemistry. For example, hydroxy groups, in order to prevent unwanted side reactions, sometimes need to be protected (e.g., converted to ethers or esters) during chemical reactions at other sites in the molecule. The hydroxy protecting group is then removed to provide the free hydroxy group. Similarly, amino groups and carboxylic acid groups can be protected (e.g., by derivatization) to protect them against unwanted side reactions. Typical protecting groups, and methods for attaching and cleaving them, are described fully in the above incorporated references by T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996).

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40, as well as other references known to one skilled in the art. It should be appreciated that various modifications to known synthetic reaction schemes can be made and will be suggested to one skilled in the art having referred to the compounds disclosed herein.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Pharmaceutical Composition

The compounds of the present invention have a variety of physiological properties including modulating, preferably reducing or inhibiting, TGF-β activity. In particular, some compounds of the present invention are found to be antagonists of TGF-β ligand. Therefore, they can be used in a variety of applications where modulating TGF-β activity in a subject is desired. For example, compounds of the present invention can be used to treat a TGF-mediated disease in a subject. Exemplary diseases that are mediated by TGF include, but are not limited to, cancer, glomerulonephritis, diabetic nephropathy, hepatic fibrosis, pulmonary fibrosis, arterial scleroderma, excessive scarring, hyperplasia, restenosis, scleroderma, and dermal scarring. In addition, since TGF-β also stimulates immune systems, compounds of the present invention may be used to stimulate a patient's immune system as well as to reduce the risk of cancer in a patient. Other uses include reduction of scarring for glaucoma filtration surgery or eye surgeries. Compounds of the present invention can also be used to inhibit fibroblast activation, collagen induction, myocardial fibrosis, and reverse disstolic dysfunction in pressure-overloaded patients.

The compounds of the present invention can be administered to a patient to achieve a desired physiological effect. Preferably the patient is an animal, more preferably a mammal, and most preferably a human. The compound can be administered in a variety of forms adapted to the chosen route of administration, i.e., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

The active compound can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparation can contain at least 0.1% of active compound. The percentage of the compositions and preparation can, of course, be varied and can conveniently be between about 1 to about 10% of the weight of the unit. The amount of active compound in such therapeutically useful compositions is such that a suitable dosage will be obtained. Preferred compositions or preparations according to the present invention are prepared such that an oral dosage unit form contains from about 1 to about 1000 mg of active compound.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens a preservatives, a dye and flavoring such as cherry or orange flavor. Of course, any material used in preparing any dosage unit form should be pharmaceutically pure and substantially non-toxic in the amounts employed. In addition, the active compound can be incorporated into sustained-release preparations and formulation.

The active compound can also be administered parenterally. Solutions of the active compound as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the active compound in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The therapeutic compounds of the present invention can be administered to a mammal alone or in combination with pharmaceutically acceptable carriers, as noted above, the proportion of which is determined by the solubility and chemical nature of the compound, chosen route of administration and standard pharmaceutical practice.

The physician can determine the dosage of the present therapeutic agents which will be most suitable for prophylaxis or treatment and it will vary with the form of administration and the particular compound chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

According to the invention, in the treatment of a TGF-related disease state, a compound of the present invention, as described herein, whether alone or as part of a pharmaceutical composition may be combined with another compound(s) of the present invention and/or with another therapeutic agent(s). Examples of suitable therapeutic agent(s) include, but are not limited to, standard non-steroidal anti-inflammatory agents (hereinafter NSAID's) (e.g, piroxicam, diclofenac), propionic acids (e.g., naproxen, flubiprofen, fenoprofen, ketoprofen and ibuprofen), fenamates (e.g., mefenamic acid, indomethacin, sulindac, apazone), pyrazolones (e.g., phenylbutazone), salicylates (e.g., aspirin), COX-2 inhibitors (e.g., celecoxib, valdecoxib, rofecoxib and etoricoxib), analgesics and intraarticular therapies (e.g., corticosteroids) and hyaluronic acids (e.g., hyalgan and synvisc), anticancer agents (e.g., endostatin and angiostatin), cytotoxic drugs (e.g., adriamycin, daunomycin, cis-platinum, etoposide, taxol, taxotere),alkaloids (e.g., vincristine), and antimetabolites (e.g., methotrexate), cardiovascular agents (e.g., calcium channel blockers), lipid lowering agents (e.g., statins), fibrates, beta-blockers, ACE inhibitors, angiotensin-2 receptor antagonists and platelet aggregation inhibitors, CNS agents (e.g., as antidepressants (such as sertraline)), anti-Parkinsonian drugs (e.g., deprenyl, L-dopa, Requip, Mirapex), MAOB inhibitors (e.g., selegine and rasagiline), comP inhibitors (e.g., Tasmar), A-2 inhibitors, dopamine reuptake inhibitors, NMDA antagonists, Nicotine agonists, Dopamine agonists and inhibitors of neuronal nitric oxide synthase), anti-Alzheimer's drugs (e.g., donepezil, tacrine, COX-2 inhibitors, propentofylline or metryfonate), osteoporosis agents (e.g., roloxifene, droloxifene, lasofoxifene or fosomax), and immunosuppressant agents (e.g., FK-506 and rapamycin).

Additional objects, advantages, and novel features of this invention will become apparent to those skilled in the art upon examination of the following examples thereof, which are not intended to be limiting.

### EXAMPLES

### Example 1

Immulon 1B or 2 HB 96 well plates (Dynex Technologies, Inc., Chantilly, Va.) were coated with 100 μL/well of 500 ng/mL TβRII (R&D Systems, Minneapolis, Minn.) in phosphate buffered saline overnight at 4° C. and then blocked for 1 hr with 150 μL/well of 1% bovine serum albumin, 150 mM NaCl, 100 mM tris-HCl, pH 7.6. Following washing with PBS/0.05% Tween 20 (wash buffer, i.e., “WB”) samples were serially diluted in 1 mg/mL BSA, 0.15 M NaCl, 100 mM tris-HCl, pH 7.6, 0.05% Tween 20 containing 5 ng/mL of 125, labeled TGF-β1 as described by Frolik et al., in J. Biol. Chem., 1984, 259, 10995-11000. Samples were incubated in the wells with TβRII for one hour at room temperature and then the plates were washed with WB. Individual wells were separated and counted.

Luciferase Assays

The pTARE luciferase reporter system was purchased from Stratagene (La Jolla, Calif.) and transfected into CHO (Chinese Hamster Ovary) cells that were purchased from ATCC (American Type Culture Collection). The plasmid pSV2neo was co-transfected with pTARE at a 40 fold lower concentration to allow for selection of colonies that received plasmid DNA using G418. Luciferase assays were performed as described using Luciferase assay system and reporter lysis buffer (Promega, Madison, Wis.). Luciferase was quantitated using a luminometer (Turner Designs, Inc., Sunnyvale, Calif.).

Growth Inhibition Assays

Mv1Lu cells were purchased from ATCC. Growth inhibition assays for Mv1Lu and CHO cells were performed as described by Danielpour et al., in J. Cell. Physiol., 1989, 138, 79-86, using 0.2% fetal bovine serum to limit binding proteins present in serum.

Chemical Screening

To identify novel organic chemicals that block TGF-B activity, about 30,000 chemicals from the National Cancer Institute Developmental Therapeutics program open repository were screened. This repository contains about 140,000 chemicals. For screening, soluble TBRII was attached to an immulon 2 HB plate and then blocked with BSA. About 1 μL of each test chemical was diluted from the stock plate into 125 μL of dilution buffer containing 5 ng/mL I-125 labeled TGF-β1. In some cases, 1 μL of four chemicals each was combined to allow higher throughput. The diluted chemical(s) was then added to the plate containing TBRII and incubated for one hour. A standard curve was included with non-radioactive TGF-β1 ranging from 500 pg/μL to 15.6 pg/μL. Chemicals that blocked I-1 25 TGF-β binding were re-tested and used for further analysis. Table 1 is a list of some of the compounds that were identified to decrease binding of labeled TGF-β1 to TβRII in the binding assay.

TABLE 1
List of some of the compounds shown to have TGF-β modulating activity.

| | | | | | |
|---|---|---|---|---|---|
| NSC 103773 | NSC 201863 | NSC 5113 | NSC 119886 | NSC 303769 | NSC 12492 |
| NSC 7215 | NSC 357777 | NSC 15596 | NSC 128884 | NSC 657704 | NSC 306960 |
| NSC 134149 | NSC 169936 | NSC 9608 | NSC 119915 | NSC 293161 | NSC 148354 |
| NSC 119911 | NSC 143101 | NSC 7520 | NSC 361672 | NSC 119889 | NSC 119910 |
| NSC 134137 | NSC 23217 | NSC 119913 | NSC 134148 | NSC 13480 | — |

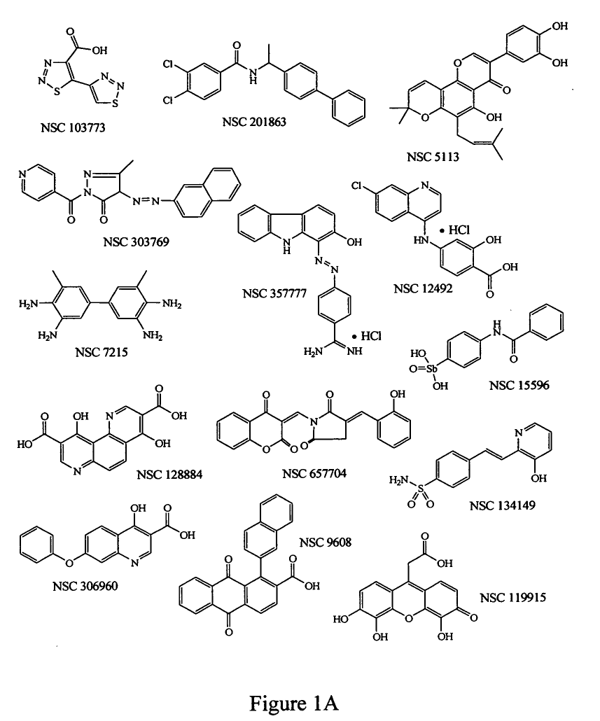

Some of the structures of compounds listed in Table 1 are shown in FIGS. 1A-1C. In addition, structures for these compounds can also be found at: http://dtp.nci.nih.gov/dtpstandard/ChemData/index.jsp. As can be seen in FIGS. 1A-1C, compounds NSC 119915, NSC 119911, NSC119910, NSC 119913, and NSC 119889 are structurally related.

### Example 2

Compounds of Table 1 (see Example 1 above) were further tested for their ability to modulate TGF-β1 activity. Briefly, the CHO cells carrying the plasmid pTARE-Luc were treated either with each compound individually (1 μL/mL) or with added TGF-β1 (25 ng/mL). Without being bound by any theory, it is believed that testing the compound by itself in this assay allows the identification of candidate TβRII agonists while testing the chemical in the presence of TGF-β1 allows the identification of candidate TGF-β1 antagonists.

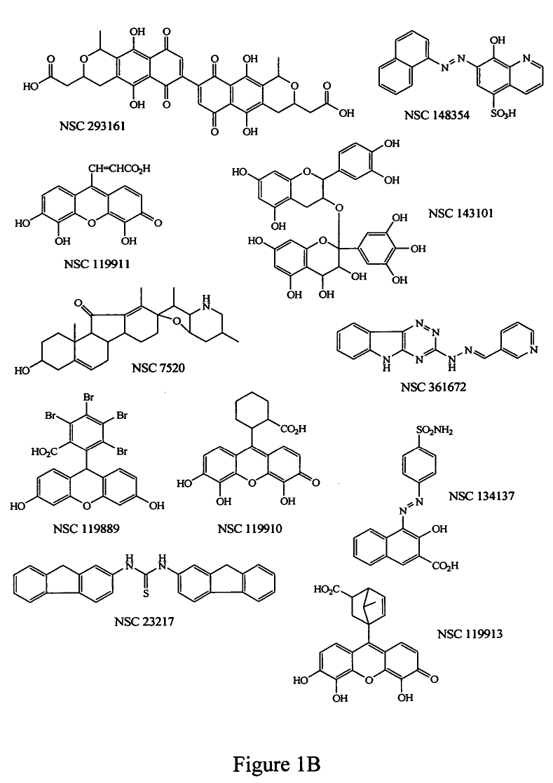

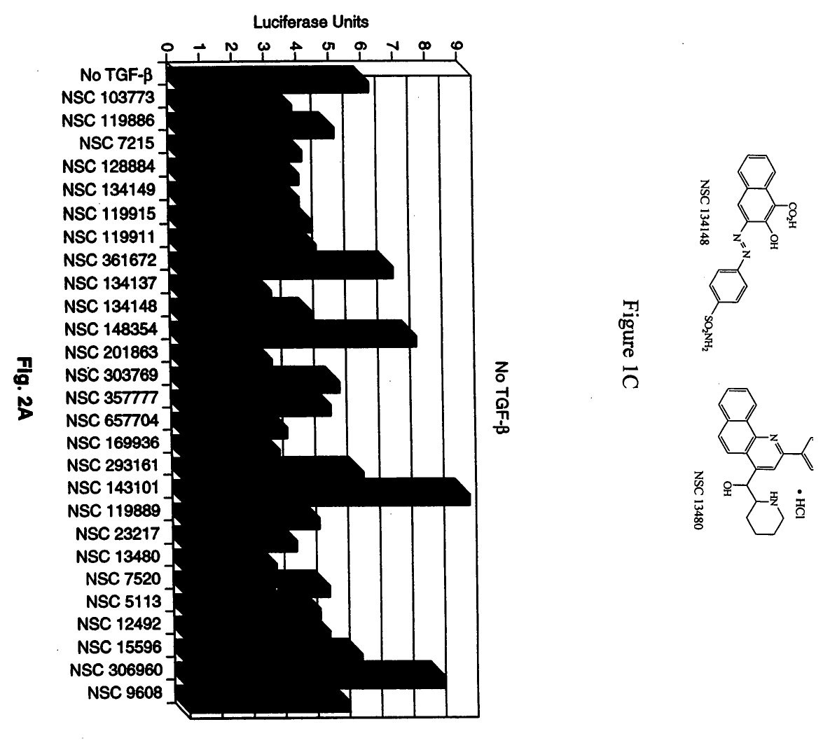

FIG. 2A shows that none of the compounds stimulated luciferase activity in the absence of TGF-β1. FIG. 2A also shows that luciferase activity in the absence of TGF-β1 in this assay is approximately 5 relative units. FIG. 2B shows that the addition of TGF-β1 to the assay stimulated production of luciferase approximately 10 fold to 50 relative units. As shown in FIG. 2B, some compounds had no effect on luciferase activity, some compounds markedly increased the total amount of luciferase activity and some compounds markedly decreased luciferase activity. Without being bound by any theory, it is believed that compounds that increase total luciferase activity may be interacting with TGF-β receptors to increase binding of TGF-β1 to the receptor or these compounds may be interacting with other physiological pathways that stimulate luciferase without a direct interaction with TβRII or the TGF-β signaling pathway. It is also believed that compounds that decrease luciferase activity may be blocking the interaction between TGF-β1 and TβRII, blocking interaction with other pathways required for luciferase expression and activity, or by cell death.

### Example 3

Chinese hamster ovarian (i.e., CHO) cells were cultured in the presence of compounds and tested for growth in the presence of tritiated thymidine. Table 2 shows the amount of radioactivity incorporated into CHO cells in the absence or presence of NSC 119889 for two different experiments. The reported value is the average of duplicate assays. From the two experiments, it is believed that the compound did not substantially inhibit growth of CHO cells and that the marked reduction in luciferase activity is due to blocking of TGF-β1 activity.

TABLE 2

| | Untreated | NSC 119889 |
|---|---|---|
| Experiment 1 | 1026 | 1475 |
| Experiment 2 | 1288 | 936 |

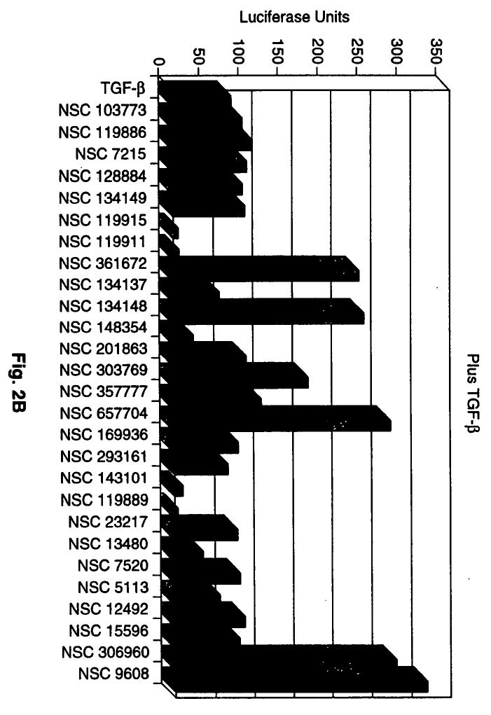

Further experiments were done to investigate toxicity of the compounds using DLD-1 colon cancer cells since these cells lack a functional TGF-β signaling pathway. FIG. 3 shows that the compounds moderately stimulated growth of DLD-1 cells.

### Example 5

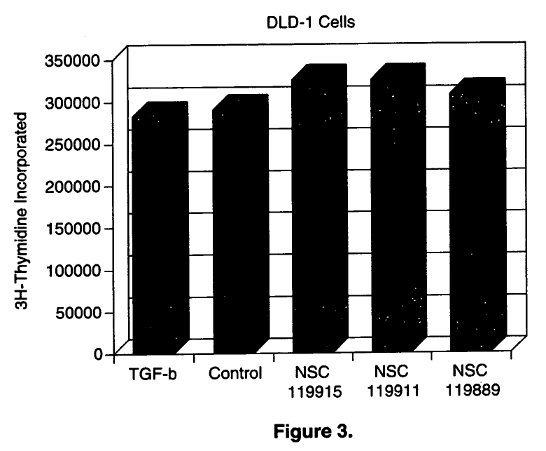

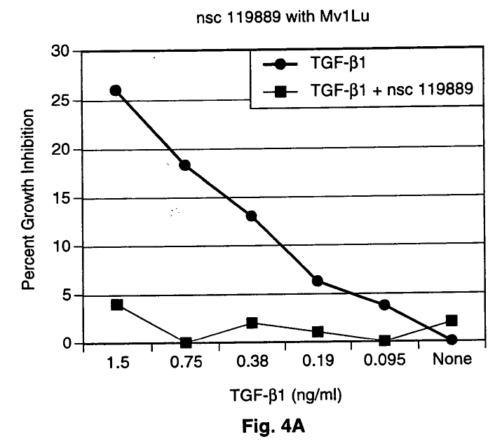

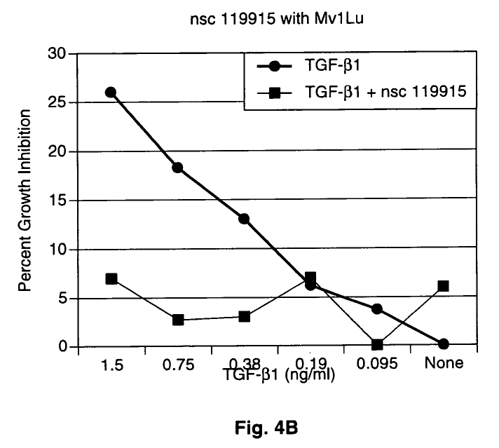

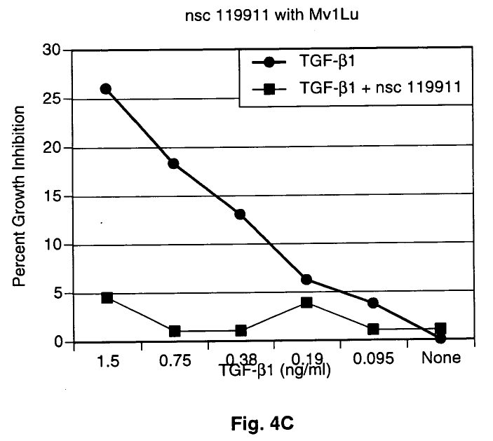

Mv1Lu cells were cultured in the presence of a standard TGF-β1 dilution curve starting at 1.5 ng/mL and serially diluted to 0.047 ng/mL or in the presence of TGF-β1 (1.5 ng/mL serially diluted to 0.095 ng/mL) with the compound. For each tested compound, one assay contained only the compound and no added TGF-β1. As shown in FIG. 4A, compound NSC 119889 blocks growth inhibition of TGF-β1. Similarly, FIG. 4B shows that compound NSC 119915 blocks growth inhibition of TGF-β1. FIG. 4C shows that compound NSC 119911 blocks growth inhibition by TGF-β1. In contrast, FIG. 4D shows that the control test compound NSC 306960, which did not inhibit TGF-β1 induction of luciferase activity, did not block the growth inhibition activity of TGF-β1.

### Example 6

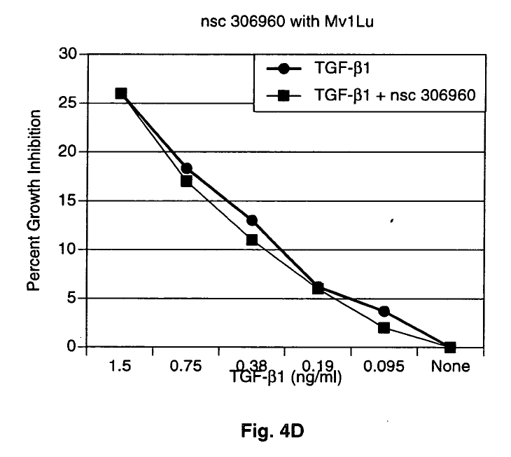

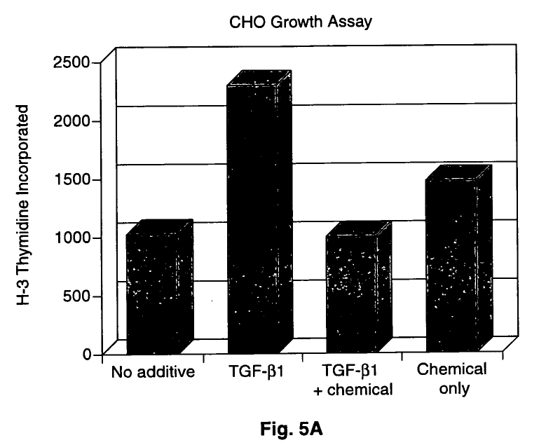

FIG. 5 shows stimulation of growth for CHO cells in the presence of TGF-β1. This stimulation was blocked by the addition of NSC 119889. FIG. 5A shows results using 1 μL of blocking compound and FIG. 5B shows results using 2 μL of blocking compound.

### Example 7

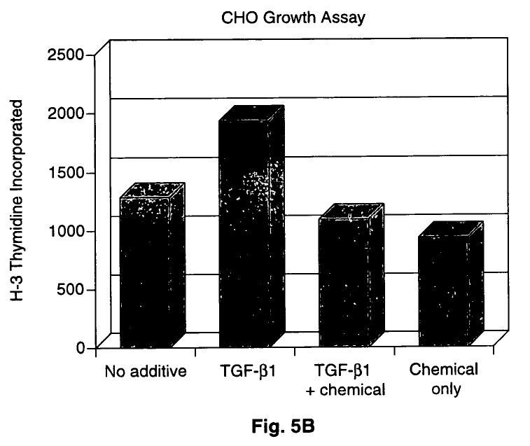

FIG. 6 shows a binding competition curve for various compounds vs TGF-β1 for soluble TβRII. In this assay, soluble receptor was attached to the plate and incubated with I-125 TGF-β1 in the presence of increasing concentration of non-labeled TGF-β1 or dilutions of the compounds. The resulting curves demonstrate that TGF-β1 is an effective competitor for TβRII in the nanomolar range whereas the compounds are effective competitors in the micromolar range.

### Example 8

Compounds NSC 119910, NSC 119911 and NSC 119915 form precipitates when diluted into water at room temperature. These precipitates are black in color and can easily be visualized after centrifugation at 17,000×G. In contrast, NSC 119889 and NSC 119913 form less or no precipitate under the same condition.

About 10 μL of ¹²⁵I labeled TGF-β1 (22.78 μCi/ml, 6.3 pmole/ml) was incubated with 0.5 μL of the compound in 100 μL of DMEM, 0.2% FBS overnight and then centrifuged. Ninety μL of the supernatant was counted and the results are shown in Table 3. Precipitation of test compounds did not remove ¹²⁵I from solution demonstrating that reduction of TGF-β1 in the assays described above was not due to precipitation of the TGF-β1. However, for compound NSC 119889 there was approximately a 40% increase in the amount of ¹²⁵I that remained in solution relative to the control. While this might be due to the compound decreasing adherence of TGF-β1 to the microcentriflige tube, the lack of precipitation of the ¹²⁵I labeled TGF-β1 is consistent with the compounds blocking interaction of TGF-β1 with the receptor possibly by direct interaction with the receptor.

TABLE 3

| | Amount of ¹²⁵I labeled | | | |
|---|---|---|---|---|
| | TGF-β1 in solution | | | Solubility |
| Control | 30718 | 18053 | 21390 | |
| NSC 119889 | 38324 | 27159 | 29239 | no ppt |
| NSC 119910 | | 24924 | 20587 | ppt |
| NSC 119911 | 33990 | 25748 | 24534 | ppt |
| NSC 119913 | | 27614 | 27960 | no ppt |
| NSC 119915 | 33660 | 25308 | 20770 | ppt |
| NSC 143101 | 34567 | | | no ppt |
| NSC 306960 | 32155 | | | no ppt |

### Example 9

Experiments were next done to determine if the compounds also block the activity of activin, a protein structurally related to TGF-β1 that functions through receptors structurally related to TβRII.

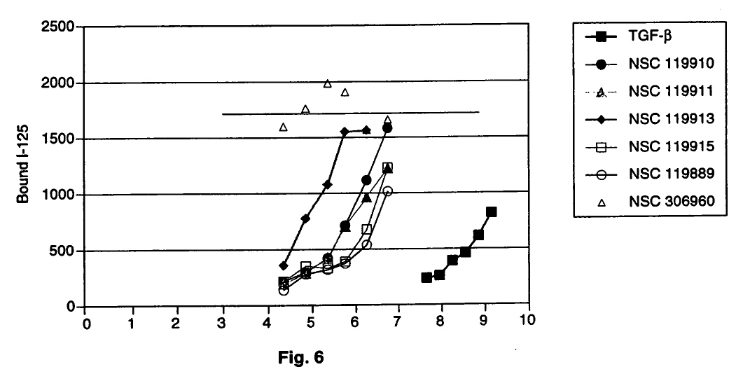

T47D breast cancer cells were incubated with tritiated thymidine and (1) media, (2) media containing activin, (3) media containing only the compound, or (4) media containing activin, and the compound. The cell growth was measured and the results are shown in FIG. 7 (the values in FIG. 7 were adjusted by subtracting 15,000 counts to allow better separation on the graph between the respective values).

### Example 10

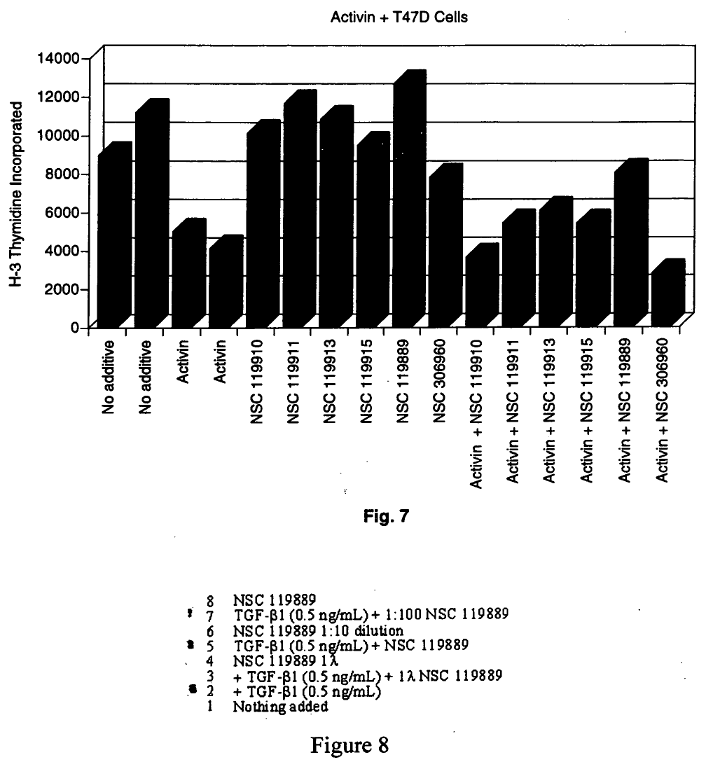

FIG. 8 shows a northern blot from Mv1Lu cells in the absence of TGF-β, presence of TGF-β, presence of TGF-β and the compound, compound only, TGF-β and 1:10 dilution of the compound, and TGF-β and 1:100 dilution of the compound. This figure shows that the compound blocks TGF-β dependent increase in expression of plasminogen activator inhibitor-1 in mink lung epithelial cells. In addition, FIG. 8 shows the marked difference in expression pattern with treatment. It is believed that there is gene expression in the control lanes and the treated lanes, but the contrast of the figure has been adjusted to emphasize and clearly show the difference in gene expression when treated with the compound.

### Example 11

The compound NSC 119889 and related molecules also block soluble TGF-β receptor type III (TβRIII). TβRIII binds TGF-β1 and TGF-β2 and is involved in presenting TGF-β2 to TβRII. The compound NSC 119889, when incubated in the presence of iodine 125 (i.e., ¹²⁵I) labeled TGF-β1, blocks binding of the ¹²⁵I labeled TGF-β1 to TβRIII. TβRIII was purchased from R&D Systems (Minneapolis, Minn.). It was used at concentration of 500 ng/mL in the soluble receptor assay when lmrulon 2HB plates were used and at 1 μg/mL when Immulon 1B plates were used. Table 4 shows the results of the soluble receptor assay for some of the compounds of the present invention.

TABLE 4

| | Amount of ¹²⁵I labeled TGF-β1 bound to TβRIII |
|---|---|
| Control | 25048 |
| NSC 119915 | 726 |
| NSC 119911 | 769 |
| NSC 119889 | 746 |

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references disclosed herein are incorporated by reference in their entirety.

What is claimed is:

1. A method of modulating TGF-β activity in a subject comprising administering to the subject an effective amount of a compound of the formula: 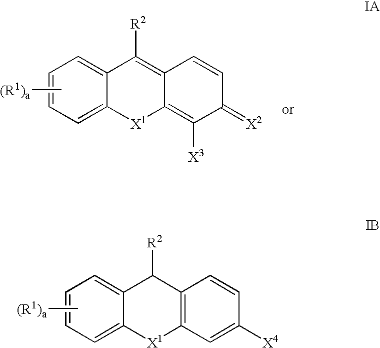
or a salt, prodrug, tautomer, hydrate, solvate, or stereoisomer thereof, wherein
a is an integer from 0 to 4;
each R¹ is independently hydroxy, alkoxy, halide, alkyl, cyano, nitro, amino, monoalkylamino, dialkylamino, or carboxy;
R² is aryl, cycloalkyl, alkenyl, or alkyl, each of which is optionally substituted;
each of X¹ and X² is independently O, S or NR³,
wherein
R³is hydrogen or alkyl;
X³ is hydrogen, alkoxy, alkyl, hydroxy, halide, amino, monoalkylamino, or dialkylamino; and
X⁴ is alkoxy, hydroxy, halide, amino, monoalkylamino, or dialkylamino.

2. The method of claim 1, wherein a is 1 or 2.

3. The method of claim 2, wherein each R¹ is independently hydroxy or alkoxy.

4. The method of claim 3, wherein X¹ is O.

5. The method of claim 4, wherein the compound is of the formula: 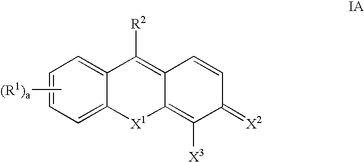
wherein R², X² and X³ are those defined in claim 1.

6. The method of claim 5, wherein X² is O.

7. The method of claim 6, wherein X³ is hydroxy or alkoxy.

8. The method of claim 6, wherein R² is substituted aryl.

9. The method of claim 4, wherein the compound is of the formula: 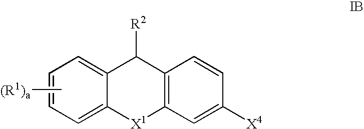
wherein R² and X⁴ are those defined in claim 1.

10. The method of claim 9, wherein X⁴ is hydroxy or alkoxy.

11. The method of claim 10, wherein R² is substituted cycloalkyl, substituted alkenyl, or substituted alkyl.

12. A method of treating a TGF-mediated disease comprising administering to a patient suffering from a TGF-mediated disease a therapeutically effective amount of a TGF-β antagonist.

13. The method of claim 12, wherein the TGF-mediated disease is selected from the group consisting of cancer, glomerulonephritis, diabetic nephropathy, hepatic fibrosis, pulmonary fibrosis, arterial scleroderma, excessive scarring, hyperplasia, restenosis, scleroderma, and dermal scarring.

14. The method of claim 12, wherein the TGF-β antagonist is a compound of the formula: 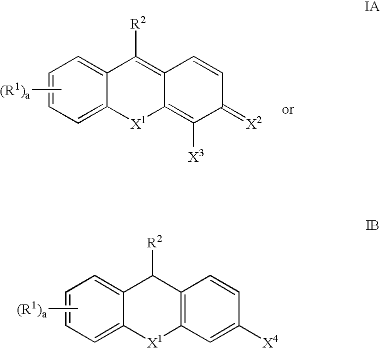
or a salt, prodrug, tautomer, hydrate, solvate, or stereoisomer thereof,
wherein
a is an integer from 0 to 4;
each R¹ is independently hydroxy, alkoxy, halide, alkyl, cyano, nitro, amino, monoalkylamino, dialkylamino, or carboxy;
R² is aryl, cycloalkyl, alkenyl, or alkyl, each of which is optionally substituted;
each of X¹ and X² is independently O, S or NR³,
wherein
R³is hydrogen or alkyl;
X³ is hydrogen, alkoxy, alkyl, hydroxy, halide, amino, monoalkylamino, or dialkylamino; and
X⁴ is alkoxy, hydroxy, halide, amino, monoalkylamino, or dialkylamino.

15. A method for stimulating a patient's immune system, said method comprising administering a therapeutically effective amount of a TGF-β antagonist to the patient.

16. The method of claim 15, wherein the TGF-β antagonist is a compound of the formula: 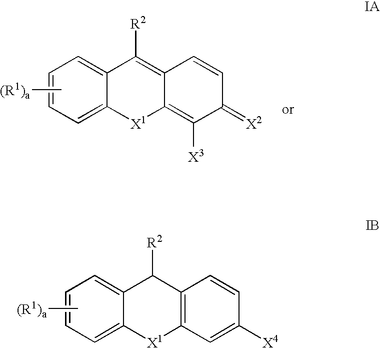
or a salt, prodrug, tautomer, hydrate, solvate, or stereoisomer thereof,
wherein
a is an integer from 0 to 4;
each R¹ is independently hydroxy, alkoxy, halide, alkyl, cyano, nitro, amino, monoalkylamino, dialkylamino, or carboxy;
R² is aryl, cycloalkyl, alkenyl, or alkyl, each of which is optionally substituted;
each of X¹ and X² is independently O, S or NR³,
wherein
R³is hydrogen or alkyl;
X³ is hydrogen, alkoxy, alkyl, hydroxy, halide, amino, monoalkylamino, or dialkylamino; and
X⁴ is alkoxy, hydroxy, halide, amino, monoalkylamino, or dialkylamino.

17. A method for reducing a risk of cancer in a subject, said method comprising administering a therapeutically effective amount of a TGF-β modulator to the subject to significantly reduce the TGF-β activity.
